(19) United States
(12) Patent Application Publication
(10) Pub. No.: US 2022/0375572 A1
(43) Pub. Date: Nov. 24, 2022

(54) ITERATIVE GENERATION OF INSTRUCTIONS FOR TREATING A SLEEP CONDITION

(71) Applicant: HypnoCore Ltd., Petach-Tikva (IL)

(72) Inventors: Yuval Eliezer ALTMAN, Tzur Yitzhak (IL); Shulamit EYAL, Givat Shmuel (IL); Armanda Lia BAHARAV, Tel-Aviv (IL)

(73) Assignee: HypnoCore Ltd., Petach-Tikva (IL)

(21) Appl. No.: 17/322,964

(22) Filed: May 18, 2021

(51) Int. Cl. G16H 20/70; G06N 20/00; G16H 10/60

(52) U.S. Cl. CPC G16H 20/70; G06N 20/00; G16H 10/60

(57) ABSTRACT

There is provided a method of generating outcomes for improving a sleep condition, comprising: iterating over sleep sessions: accessing previously generated instructions for treatment for improvement of the sleep condition for a recent sleep session obtained as a previous outcome of a machine learning model, accessing sleep-parameters computed for the recent sleep session, feeding the sleep-parameters and previously generated instructions into the machine learning model, and obtaining as an outcome of the machine learning model, instructions for presentation on a user interface indicating treatment for improvement of the sleep condition of the target individual for a new sleep session following the recent sleep session, wherein a new set of sleep-parameters are accessed for the new sleep session, for feeding into the machine learning model in combination with the instructions for the new sleep session, for obtaining another set of instructions for another sleep session following the new sleep session.

### FIELD AND BACKGROUND OF THE INVENTION

The present invention, in some embodiments thereof, relates to treatment of sleep conditions and, more specifically, but not exclusively, to systems and methods for generation of outcomes (e.g., instructions) for treating and/or evaluating the sleep condition.

Sleep is a highly complex physiological and psychological state, a basic biological necessity. According to a variety of surveys and published scientific articles, the majority of the adult population suffers from at least one sleep-related condition, issue, disturbance, and/or symptoms, which may impair the quality and/or duration of their sleep. Consecutively, these impairments lead to considerable increase in health-related risks, varied accidents, reduced productivity, lower wellbeing, and more. Different sleep improvement approaches have been developed to try and improve the amount and/or quality of sleep the person receives.

### SUMMARY OF THE INVENTION

According to a first aspect, a computer implemented method of generating outcomes for treatment for improving a sleep condition in a target individual, comprises: iterating over a plurality of sleep sessions: accessing a plurality of previously generated instructions for treatment for improvement of the sleep condition for a recent sleep session obtained as a previous outcome of a machine learning model, accessing a plurality of sleep-parameters computed for the recent sleep session, feeding the plurality of sleep-parameters and the plurality of previously generated instructions into the machine learning model, and obtaining as an outcome of the machine learning model, instructions for presentation on a user interface indicating treatment for improvement of the sleep condition of the target individual for a new sleep session following the recent sleep session, wherein a new set of plurality of sleep-parameters are accessed for the new sleep session, for feeding into the machine learning model in combination with the instructions for the new sleep session, for obtaining another set of instructions for another sleep session following the new sleep session.

According to a second aspect, a computer implemented method of training a machine learning model for generating outcomes for treatment for improvement of a sleep condition in a target individual, comprises: obtaining records for each of a plurality of sample individuals, each record including sample sleep-parameters, previously generated instructions for treatment for improvement of a respective sleep condition for a recent sleep session, and a sleep-state indicating a state of a respective sleep condition of the respective sample individual, labelling each record with a ground truth indication of instructions for improving the respective sleep condition of the respective sample individual during a subsequent sleep session, generating a training dataset that includes the labelled records, and training the machine learning model on the training dataset for generating instructions for presentation on a user interface indicating treatment for improvement of a target sleep condition of a target individual for a new sleep session following a recent sleep session in response to an input of sleep-parameters and previously generated instructions, wherein the machine learning model is iteratively trained by updating the records using a new set of sleep-parameters accessed for the new sleep session, and using the instructions for the new sleep session obtained as outcomes of the machine learning model for the recent sleep session.

According to a third aspect, a system for generating outcomes for treatment for improvement of a sleep condition in a target individual, comprises: at least one hardware processor executing a code for: iterating over a plurality of sleep sessions: accessing a plurality of previously generated instructions for treatment for improvement of the sleep condition for a recent sleep session obtained as a previous outcome of a machine learning model, accessing a plurality of sleep-parameters computed for the recent sleep session, feeding the plurality of sleep-parameters and the plurality of previously generated instructions into the machine learning model, and obtaining as an outcome of the machine learning model, instructions for presentation on a user interface indicating treatment for improvement the sleep condition of the target individual for a new sleep session following the recent sleep session, wherein a new set of plurality of sleep-parameters are accessed for the new sleep session, for feeding into the machine learning model in combination with the instructions for the new sleep session, for obtaining another set of instructions for another sleep session following the new sleep session.

In a further implementation form of the first, second, and third aspects, the outcome is generated by an aggregation of weights of internal settings of the machine learning model set according to the feeding, and penalty functions applied to the weights.

In a further implementation form of the first, second, and third aspects, further comprising: accessing a sleep-state for the recent sleep session, the sleep-state indicating a state of the sleep condition of the target individual obtained as an outcome of a sleep application selected from a plurality of sleep applications, and wherein feeding further comprises feeding the sleep-state into the machine learning model, wherein a new sleep-state is accessed for the new sleep session from a new selection of the sleep application, for feeding into the machine learning model.

In a further implementation form of the first, second, and third aspects, the sleep application is selected from a group consisting of: a sleep evaluation application, a sleep improvement application, a sleep monitoring application, and a sleep maintenance application.

In a further implementation form of the first, second, and third aspects, further comprising monitoring interaction of the target user with the sleep application and/or with a graphical user interface (GUI) presenting the instructions, and wherein feeding further comprises feeding into the machine learning model, the previously monitored interaction of the target user.

In a further implementation form of the first, second, and third aspects, obtaining comprises obtaining as the outcome of the machine learning model, a presentation format selected from a plurality of candidate presentation formats, for presenting the instructions for improving the sleep condition, selected from a group consisting of: push notification, email, voice message, video message, animation, presentation, and text message, wherein feeding further comprises feeding the previously obtained presentation format into the machine learning model.

In a further implementation form of the first, second, and third aspects, the machine learning model is trained on a training dataset that includes records for a plurality of sample individual, each record for each respective sample individual including sample sleep-parameters and previously generated instructions for improving sleep for a recent sleep session labelled with a ground truth indication of instructions for generating outcomes for improving a respective sleep condition of the respective sample individual during a new sleep session.

In a further implementation form of the first, second, and third aspects, further comprising extracting a plurality of features from the plurality of sleep-parameters, wherein the plurality of features are fed into the customized machine learning model and/or used by the sleep application to generate the sleep-state.

In a further implementation form of the first, second, and third aspects, further comprising accessing a set of characteristics of the target user denoting the target user's daytime behavior and/or demographic parameters, and wherein feeding further comprises feeding into the machine learning model, the set of characteristics.

In a further implementation form of the first, second, and third aspects, feeding into the machine learning model comprises running data fed into the machine learning model through a plurality of decision trees each comprising a set of predefined rules and/or a sub-processing code, wherein each decision tree terminates in a respective neuron that outputs an active category of a binary indication when conditions in the respective decision tree are satisfied, the respective neuron is mapped to at least one output that generates the outcome of the instructions for improving the sleep condition.

In a further implementation form of the first, second, and third aspects, further comprising: multiplying neurons that output the active category by a set of weights to obtain a weighted set of active neurons, wherein the set of weights are at least one of: prefixed, determined by a weighting function that receives data fed into the machine learning model as input, and previously learned, wherein the set of weights are at least one of: defined per neuron, defined per neuronal group comprising a set of neurons congregated based on a certain characteristic and/or set of characteristics, per basket of a set of neuronal groups congregated based on a certain characteristic and/or set of characteristics, and combinations of the aforementioned.

In a further implementation form of the first, second, and third aspects, further comprising randomizing an order of the weighted neurons in the weighted set.

In a further implementation form of the first, second, and third aspects, further comprising: penalizing each respective weighted neuron based on neurons that were activated during a previous feeding iteration of the machine learning model that generated the outcome of the previously generated instructions, aggregating penalties computed for each respective weighted neuron to obtain a respective total neuron-penalty, and applying a transfer function on each respective total neuron-penalty.

In a further implementation form of the first, second, and third aspects, the penalizing is based on a member selected from a group consisting of: (i) whether a respective individual weighted neuron was previously activated, (ii) whether a neuronal group of which the respective individual weighted neuron is a member, includes neurons that were previously activated, (iii) whether the respective individual weighted neuron that previously activated generated a same output format of the instructions, and (iv) applying at least one additional penalty defining the outcome of the instructions.

In a further implementation form of the first, second, and third aspects, further comprising: for each basket of a set of neuron groups each of a set of neurons, selecting a first top set of neurons within lowest penalty values according to a first requirement, from the selected first top set of neurons, for each neuron group, selecting a second top set of neurons within lowest penalty values according to a second requirement, from the selected second top set of neurons, selecting a third top set of neurons within lowest penalty values according to a third requirement, wherein the outcome of the instructions is generated according to the third top set of neurons.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system for generating outcomes for treating a sleep condition in a target individual based on previously generated outcomes for a recent sleep session and sleep-parameters of the recent sleep session, in accordance with some embodiments of the present invention;

FIG. 2 is a flowchart of a method of generating outcomes for treating a sleep condition in a target individual based on previously generated outcomes for a recent sleep session and sleep-parameters of the recent sleep session, in accordance with some embodiments of the present invention;

FIG. 3 is a flowchart of a method of training a machine learning model for generating outcomes for treating a sleep condition in a target individual based on previously generated outcomes for a recent sleep session and sleep-parameters of the recent sleep session, in accordance with some embodiments of the present invention; and

FIG. 4 is a flowchart of a method of inference by a machine learning model using neuron and/or decision trees, in accordance with some embodiments of the present invention.

### DESCRIPTION OF SPECIFIC EMBODIMENTS OF THE INVENTION

The present invention, in some embodiments thereof, relates to treatment of sleep conditions and, more specifically, but not exclusively, to systems and methods for generation of outcomes (e.g., instructions) for treating and/or evaluating the sleep condition.

As used herein, a sleep condition may be a clinically diagnosed sleep disorder, for example, insomnia. The sleep condition may relate to one or more of the following: The sleep condition may be a subject state in individuals which are not suffering from a clinically diagnosed sleep disorder. The sleep condition may be in individuals that may or may not have a history of sleep disorders, and/or may or may not currently have a healthy sleep. The sleep condition may relate to a state of sleep, for which improvement may be desired. The sleep condition may relate to a state of sleep, for which additional insight is desired. The sleep condition may relate to a state of sleep, which may be good sleep, which the user desires to maintain. The sleep condition may be in individuals that have poor sleep hygiene and/or sleep-related habits, which may or may not impair their sleep quality and/or duration, for example, inconsistent wake up times, caffeine consumption before bedtime, alcohol consumption before bedtime, exercising prior to bedtime, suboptimal bed environment in terms of noise, temperature, light, etc. The sleep condition may be secondary related to a primary condition, optionally medical condition, for which the sleep condition is acting as a secondary condition, for example, due to pain, arthritis, cardiovascular condition, hypertension, diabetes, lung disease, allergies, depression, anxiety, post-traumatic stress disorder, or other conditions. It is noted that that the approaches described herein do not necessarily aim to aid in treating these primary conditions directly, but may aid in improvement of the secondary sleep condition, such as for any sleep-related treatment, evaluation, maintenance, monitoring etc.

As used herein, the outcomes of the machine learning model described herein for treating and/or evaluating the sleep condition may be part of a treatment of the sleep condition (e.g., disorder), and/or may not necessarily be instructions and/or may not necessarily be a part of a treatment. For example, when the target individual experiences some sleep difficulties, for which the target individual is getting treatment, the outcomes may be instructions as part of such treatment. In another example, when the target individual experiences some sleep difficulties, for which the target individual is getting treatment, the outcomes may support and accompany such treatment (without specifically instructing target individual). In yet another example, when the target individual is taking part in a sleep evaluation program, the outcomes may be instructions for completing the evaluation, and/or increasing its accuracy and reliability.

An aspect of some embodiments of the present invention relates to systems, methods, a computing device, and/or code instructions (stored on a memory and executable by hardware processors) for dynamically generating outcomes (e.g., instructions) for treating and/or evaluating a sleep condition in a target individual (also referred to herein as a user), for example, daily instructions for improving quality of sleep on the user, and/or for maintain a good quality sleep, and/or for treatment of a clinically diagnosed sleep disorder. The outcome is of a machine learning model, in response to an input of previously generated outcomes (e.g., instructions) for improving sleep for a recent sleep session obtained from the machine learning model in a previous iteration, sleep-parameters obtained for the recent sleep session, and optionally other data (as described herein). The machine learning model iteratively generates from the previously generated instructions and associated sleep-parameters which are obtained for the recent sleep session to which the generated instructions applied, to generate new instructions for a new sleep session to treat the sleep condition. By using the previous instructions and impact of the instruction on the sleep-parameters, the machine learning model iteratively generates new adjusted instructions to treat the sleep condition in the next night, and/or at other intervals, for example, multiple iterations per day, or each iteration over multiple nights (e.g., 2, 3, or a week). The iterations enable determining which previous instructions worked to treat the sleep condition and which didn't, by their impact on the sleep-parameters and/or other data. The iterations enable dynamic adjustment to changing conditions of the user, for example, stressful events, change in sleep habits, change in eating habits, and the like. The changing conditions of the user directly or indirectly impact the sleep-parameters and/or other data, enabling the machine learning model to generate new instructions that are more impactful to the user in view of the changed conditions, for treating the sleep condition.

The machine learning model may be dynamically updated based on the input of a specific user and/or outcomes generated for the specific user, to dynamically create a personalized machine learning model that is customized for the specific user.

At least some implementations of the systems, methods, apparatus, and/or code instructions described herein address the medical problem of treating a target individual suffering from a sleep condition. Sleep is a highly complex physiological and psychological state. The majority of the adult population suffers from at least one sleep-related condition, issue, disturbance, and/or symptoms, which may impair the quality and/or duration of their sleep. Consecutively, these impairments lead to considerable increase in health-related risks, varied accidents, reduced productivity, lower wellbeing, and more. The accepted standard practice and prevalent methods for aiding in such sleep-related issues are usually based on periodic meetings with an expert, e.g., a physician or a psychologist, in which both short- and long-term tasks are given. Cognitive behavioral therapy (CBT) is an example for such a solution. There is an abundant of software-based solutions in the sleep improvement field, such as mobile applications, online services, computer programs etc., which aim to assist people in resolving sleep-related issues and in improving sleep quality and/or quantity. These solutions commonly offer a similar structure of short- and long-term tasks. The challenge for such software-based solutions, which is addressed by at least some implantations described herein, relates to managing user communications. Addressing different elements of a sleep improvement program, a sleep maintenance program, a sleep assessment program, or other sleep-related programs, is a challenging task, especially when trying to avoid monotonous communication with the user.

At least some implementations of the systems, methods, apparatus, and/or code instructions described herein improve the medical field of treating sleep conditions, by providing a personalized user communication managing system, for managing multiple sleep applications, for example, sleep monitoring, assessment, improvement, maintenance, or other sleep-related services and/or programs.

At least some implementations of the systems, methods, apparatus, and/or code instructions described herein address the technical problem of managing user communication in sleep-related applications and/or digital services. At least some implementations of the systems, methods, apparatus, and/or code instructions described herein improve the technical field managing user communication in sleep-related applications and/or digital services. In at least some implementations, a solution to the technical problem is provided by, and/or the technical field is improved by, a process that manages user communication for sleep-related applications, that is flexible and/or modular, and/or suitable for many different populations of users and/or applications. Outputs including presentation format type and/or instructions for treating the sleep condition, are based on multiple input parameters, for example, users' sleep data, behavioral data, user usage and/or engagement data, and previous generate outputs. Outputs including instructions for treating the sleep condition may be ranked and/or prioritized, for selecting the most appropriate outputs for the given data and/or upcoming sleep session. Outputs may be communicated to the user, for example, as part of a mobile application, a computer program, an online service, and the like. Outputs may be provided in selected presentation formats, for example, push notifications, emails, voice messages, video messages, animations, presentations, textual messages and the like.

At least some implementations of the systems, methods, apparatus, and/or code instructions described herein improve over other approaches. For example, the improvement may be over a strictly rule-based approach, by providing a machine learning model which is trained. In another example, the improvement is over a non-feedback approach, by considering previous outputs of instructions for treatment of the sleep condition in generation of current outputs. This provides greater adaptability (e.g., to what the user best responds to) and/or reduces monotony (e.g., by varying the instructions and/or varying the format of the instructions). In yet another example, the improvement is over a conflict-resolving mechanism (where for cases in which multiple rules are satisfied for a given set of input data, another list of predefined rules may be used), by not necessarily using a predefined list of rules for resolving conflicts for cases in which multiple rules are satisfied for a given set of input data. The improvement may be achieved by applying a weighting function and a set of penalty functions, to prioritize rules and/or to resolve conflicts. The weighting function and/or penalty function may be functions of user's data, including historical data, and/or previous outputs of the algorithm.

A rule-based solution with an unlimited number of rules and an unlimited number of outputs will be a consistent and a reliable solution. However, such solution is extremely difficult to manage, as even finite amounts of rules and outputs are arduous to maintain when their quantities are great. Nevertheless, small amounts of rules and outputs will result in under fitting, as the model will be too simple—only a few situations will result in outputs, or the same outputs will be used constantly. In contrast, in at least some implementations described herein, using a set of weighting and penalty functions, as part of a feedback, it is possible to use substantially less rules, especially for prioritization and conflict-resolving tasks.

Cognitive behavioral therapy (CBT) is often used as a tool for addressing sleep disturbances, lower sleep quality and/or quantity, tendencies for such disturbances and more. These techniques usually comprise of both short- and long-term tasks. After some time, these can become quite convoluted and it is more difficult to manage the therapy-based user communications. In contrast, in at least some implementations described herein, by employing feedback, the therapy-based communication becomes more adaptive. This solution may allow to focus on the same task for a given time, or to diverse communication and address different tasks, by using a set of weighting and penalty functions of previous outputs.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

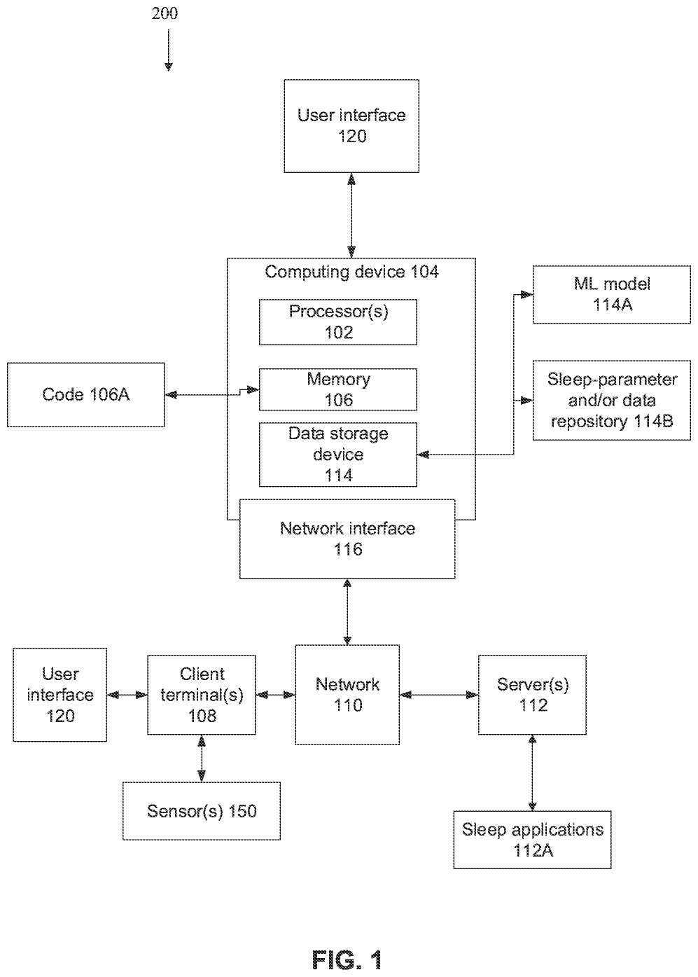

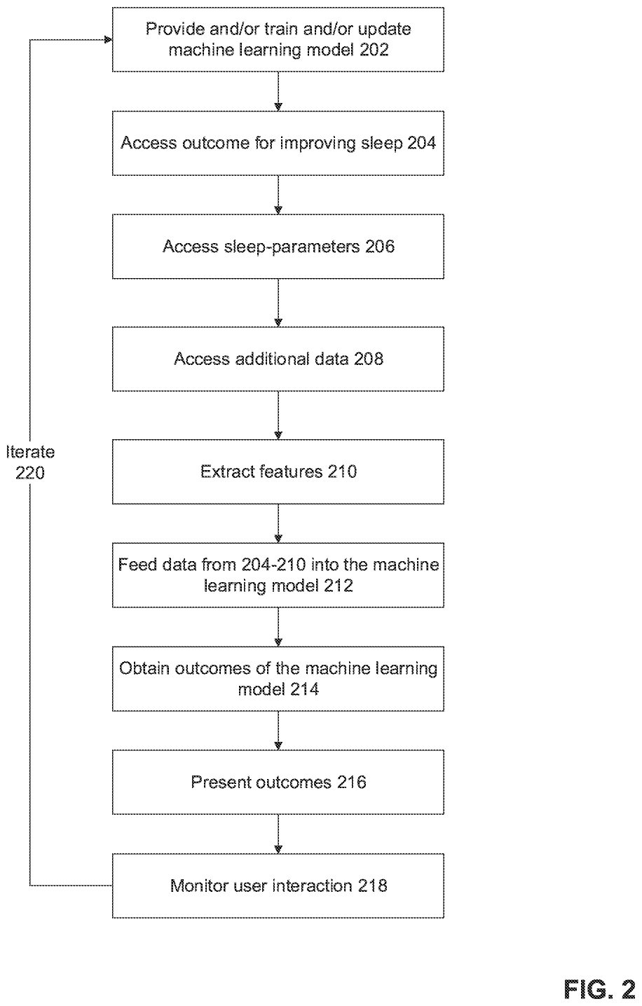

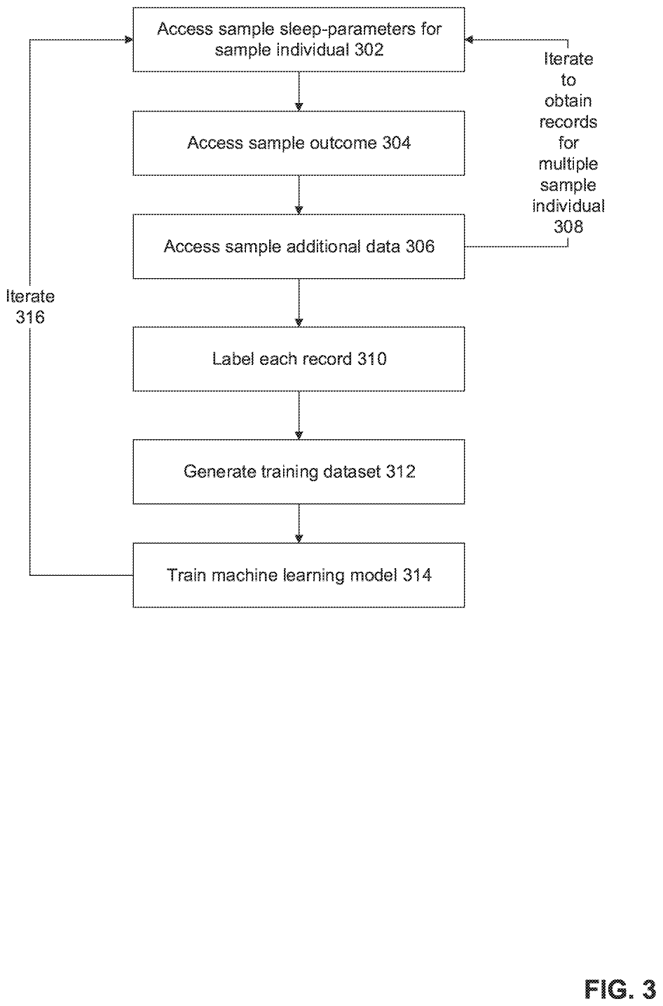

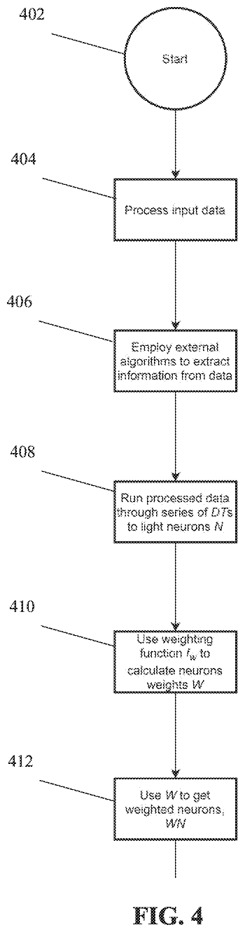

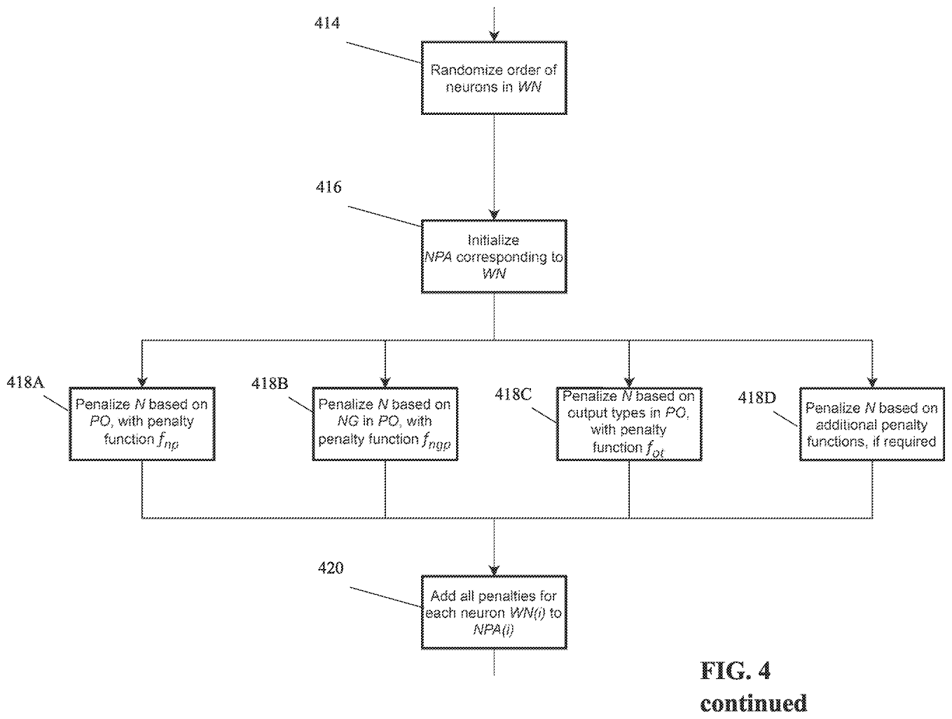

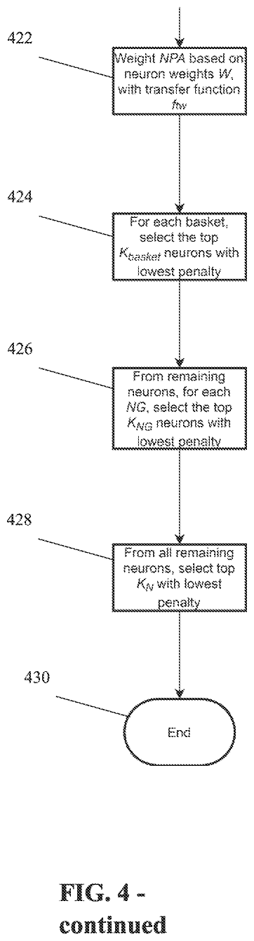

Reference is also made to FIG. 1, which is a block diagram of components of a system 100 for generating outcomes for treating a sleep condition in a target individual based on previously generated outcomes for a recent sleep session and sleep-parameters of the recent sleep session, in accordance with some embodiments of the present invention. Reference is also made to FIG. 2, which is a flowchart of a method of generating outcomes for treating a sleep condition in a target individual based on previously generated outcomes for a recent sleep session and sleep-parameters of the recent sleep session, in accordance with some embodiments of the present invention. Reference is also made to FIG. 3, which is a flowchart of a method of training a machine learning model for generating outcomes for treating a sleep condition in a target individual based on previously generated outcomes for a recent sleep session and sleep-parameters of the recent sleep session, in accordance with some embodiments of the present invention. Reference is also made to FIG. 4, which is a flowchart of a method of inference by a machine learning model using neuron and/or decision trees, in accordance with some embodiments of the present invention.

System 100 may implement the acts of the method described with reference to FIGS. 2-4, by processor(s) 102 of a computing device 104 executing code instructions stored in a memory 106 (also referred to as a program store).

Computing device 104 may be implemented as, for example one or more and/or combination of: a group of connected devices, a client terminal, a server, a virtual server, a computing cloud, a virtual machine, a desktop computer, a thin client, a network node, and/or a mobile device (e.g., a Smartphone, a Tablet computer, a laptop computer, a wearable computer, glasses computer, and a watch computer).

Multiple architectures of system 100 based on computing device 104 may be implemented. For example:
Computing device 104 may be implemented as a standalone device (e.g., kiosk, client terminal, smartphone) that include locally stored code instructions 106A that implement one or more of the acts described with reference to FIGS. 2-4. The locally stored instructions may be obtained from another server, for example, by downloading the code over the network, and/or loading the code from a portable storage device. For example, sleep-parameters (and/or data) are locally entered by a specific user using client terminal 104 and/or collected by sensors 150 connected to the computing device. The computing device locally feeds the data into the machine learning model 114A, and provides the outcome (e.g., instructions), such as for presentation on a display of the client terminal 104 of the specific user. Computing device 104 may store a personalized version of the machine learning model that is customized (e.g., trained) for the specific user.
Computing device 104 executing stored code instructions 106A, may be implemented as one or more servers (e.g., network server, web server, a computing cloud, a virtual server) that provides centralized services (e.g., one or more of the acts described with reference to FIGS. 2-4) to one or more client terminals 108 over a network 110. For example, providing software as a service (SaaS) to the client terminal(s) 108, providing software services accessible using a software interface (e.g., application programming interface (API), software development kit (SDK)), providing an application for local download to the client terminal(s) 108, providing an add-on to a web browser running on client terminal(s) 108, and/or providing functions using a remote access session to the client terminals 108, such as through a web browser executed by client terminal 108 accessing a web sited hosted by computing device 104. For example, sleep-parameters and/or data are provided from each respective client terminal 108 of each respective user to computing device 104. Computing device centrally feeds the data into the machine learning model 114A, and provides the outcome (e.g., instructions), such as for presentation on a display of each respective the client terminal 108 of each respective specific user. Computing device 104 may store a respective personalized version of the machine learning model that is customized (e.g., trained) for each specific user.

Hardware processor(s) 102 of computing device 104 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include a single processor, or multiple processors (homogenous or heterogeneous) arranged for parallel processing, as clusters and/or as one or more multi core processing devices.

Memory 106 stores code instructions executable by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Memory 106 stores code 106A that implements one or more features and/or acts of the method described with reference to FIGS. 2-4 when executed by hardware processor(s) 102.

Computing device 104 may include a data storage device 114 for storing data, for example, the machine learning model 114A described herein, and/or a sleep-parameter and/or other data repository 114B. Data storage device 114 may be implemented as, for example, a memory, a local hard-drive, virtual storage, a removable storage unit, an optical disk, a storage device, and/or as a remote server and/or computing cloud (e.g., accessed using a network connection).

Network 110 may be implemented as, for example, the Internet, a local area network, a virtual network, a wireless network, a cellular network, a local bus, a point to point link (e.g., wired), and/or combinations of the aforementioned.

Computing device 104 and/or client terminal(s) 108 may be in communication with one or more sensor(s) 150 that perform measurements for collecting sleep-parameters, as described herein in additional detail.

Computing device 104 may include a network interface 116 for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations.

It is noted that in the standalone implementation, network interface 116 is not necessarily required, as computing device 104 includes sensors 150 and/or user interface 120 in a single device that may operate without externally communication with other devices, for example, a smartphone, a kiosk, and a dedicated device.

Computing device 104 may connect using network 110 (or another communication channel, such as through a direct link (e.g., cable, wireless) and/or indirect link (e.g., via an intermediary computing unit such as a server, and/or via a storage device) with one or more of:
Remote server(s) 112 running one or more sleep applications 112A, for example, a sleep evaluation application, a sleep improvement application, a sleep monitoring application, and a sleep maintenance application. A sleep-state of the user may be obtained from the sleep application 112A.
Client terminal(s) 108, when computing device 104 is implemented as a server remotely providing the features and/or acts described with reference to FIGS. 2-4.
Sensor(s) 150 that perform measurements for collecting sleep-parameters.

Computing device 104 and/or client terminal(s) 108 include and/or are in communication with one or more physical user interfaces 120 that include a mechanism for a user to enter data (e.g., manually enter sleep-parameters) and/or view the displayed results (e.g., instructions to treat the sleep condition), within a GUI. Exemplary user interfaces 120 include, for example, one or more of, a touchscreen, a display, gesture activation devices, a keyboard, a mouse, and voice activated software using speakers and microphone.

Referring now back to FIG. 2, at 202, a machine learning model is provided and/or trained and/or updated. Exemplary machine learning models are described with reference to FIGS. 3 and/or 4.

The machine learning model may be dynamically updated based on the input of a specific user and/or outcomes generated for the specific user, to dynamically create a personalized machine learning model that is customized for the specific user. Each customized model may “learn” what instructions are most impactful to the specific user (e.g., in terms of improving the sleep condition), and/or what instructions are not impactful to the specific user, and/or changing conditions of the user (e.g., stress, sleep schedule, eating habits, coffee consumption habits, exercise regimen, and the like).

At 204, outcomes (e.g., instructions) for treating and/or improving the sleep condition (e.g. a sleep-state) for a recent sleep session are accessed. The outcomes (e.g., instructions) may be obtained as a previous outcome of the machine learning model, optionally during a previous iteration.

The outcomes are for the recent sleep session, which has already occurred to the user. For example, the recent sleep session was for a previous night's sleep. It is noted that sleep session may refer to one or more previous sleep sessions, for example, a single sleep session of the previous night's sleep, or multiple sleep session over multiple nights, for example, over the last 2, 3, or week.

Optionally, the outcomes (e.g., instructions) for multiple historical sleep sessions are accessed, for example, instructions for each of the nights of the previous week. The outcomes may be labelled with a date and/or relative location in a sequence.

Exemplary outcomes (e.g., instructions) are described with reference to 214.

At 206, sleep-parameters computed for the recent sleep session of the user are accessed.

The sleep-parameters are for the recent sleep session for which the outcomes (e.g., instructions) were related to. As such, it may be assumed that the sleep-parameters reflect the impact of application of the instructions. For example, when the instructions were for the previous night, to go to sleep 1 hour earlier than usual, the sleep-parameters obtained for the previous night reflect the impact of the user going to sleep 1 hour earlier than usual. When the user is non-compliant with the instructions, the sleep-parameter reflects the impact of the non-compliance. For example, when the user was not compliant with the instructions to go to sleep 1 hour earlier than usual, and instead went to sleep at the same time, the sleep-parameters obtained for the previous night reflect the impact of the user being non-compliant and going to sleep as usual rather than 1 hour earlier than usual.

Optionally, the sleep-parameters for multiple historical sleep sessions are accessed, for example, sleep-parameters for each of the nights of the previous week. The sleep-parameters may be labelled with a date and/or relative location in a sequence.

The sleep-parameters may be obtained for example, from sensors and/or from a client terminal of the user, such as a mobile device (e.g., smartphone, tablet, laptop). For example, sensors transmit data to the smartphone, and/or a GUI on the mobile device asks the user to enter additional data. The sleep-parameters may be locally computed and/or locally stored on the client terminal of the user, and/or data collected by the client terminal and/or sensors may be forwarded to a central server, where the central server computes the sleep-parameters and/or stores the sleep-parameters.

The target user may be an individual for which sleep-parameters are computed and/or obtained.

Sleep-parameters may be indicative of the user's sleep, for example, physiological data, behavioral data, temporal data, qualitative data, ambient data, and/or other types of data. Sleep-parameters may be subjective (e.g., asking a user to rate the quality of sleep from 1-10), and/or objective (e.g., number of times the user woke up during the night), as reported by user and/or obtained by a sensor and/or by an array of sensors, from data aggregating services for electronic records, and/or other sources. Sleep-parameters may include, for example, bedtime and wake up times, average heart rate throughout the night, total time spent in bed, sleep perception score, and more.

The sleep-parameters are obtained for each specific sleep session, for example, per night. Sleep-parameters may be acquired for multiple sleep sessions.

Sleep-parameters may be based on measurements and/or perceptions of the target individual indicative of perceived data. Measurements may be obtained from output of sensor(s) (e.g., physiological sensors, activity sensors) and/or may be obtained as data manually provided by the user via the physical user interface (e.g., via a GUI, gesture interface, and/or audio interface), for example, pressing an icon whenever caffeinated coffee is drunk. Perceptions of the target user may be obtained, for example, by the user manually entering data into the physical interface in response to one or more questions, for example, rate the quality of your sleep last night, how long did it take you to fall asleep, and how many times a night did you wake up. It is noted that the perceived data may be different than the measurements, for example, the user may perceive that it took 30 minutes to fall asleep, whereas a sensor may measure that it took the user 5 minutes to fall asleep.

Exemplary sleep-parameters include one or more of:
Total sleep time (e.g., measured in hours and/or minutes).
Sleep efficiency, denoting the ratio of the total time spend asleep (i.e., total sleep time) to the total amount dedicated to sleep (e.g., time spent in bed, including sleeping time and time trying to fall asleep, and/or time being awake after falling asleep).
Arousal index, denoting the total number of arousals (or short awakenings) divided by the total sleep time, and/or frequency of arousals (or short awakenings) events during the total sleep time.
Percent rapid eye movement (REM), denoting the percent of time during sleep spent in the REM state.
Percent deep sleep, denoting the percent of time during sleep spent in the deep sleep state.
Sleep satisfaction, denoting the perception of the target individual towards satisfaction from the night's sleep.
Day time sleepiness, denoting the perception of the target individual towards how tired/sleepy the user feels during awake time.
Sleep quality, denoting the measured value of the target individual quality of the previous night's sleep. Sleep quality is computed as an aggregation of one or more measurements performed by one or more sensors that each measure a respective night parameter. The sleep quality is represented by a single value that grades the quality of sleep of the previous night.
Night stress, denoting the amount of stress the target individual experiences at night.
Sleep fragmentation, denoting the number of fragmentation events experienced by the target individual during the sleeping period (e.g., the night).

Sleep-parameter may include data based on an activity performed by the target individual and/or a current mental state of the target individual during the day, which may be impacted by, and/or may impact, sleep during the night, for example:
Daytime naps. For example, the length of each nap, and time during the day of each nap.
Caffeine consumption. For example, the amount of caffeine ingested, and time during the day when each caffeine consumption occurred.
Alcohol intake. For example, the amount of alcohol ingested type of alcoholic beverage, and time during the day when each alcohol intake occurred.
Exercise. For example, the type of exercise, length of time of exercise, amount of calories burnt, and time during the day when exercise occurred.
Meals. For example, the amount of calories ingested, types of food eaten (e.g., food that give energy or foods that make one sleepy), and time during the day when each meal occurred.
Stress level. For example, an objective measure of stress (e.g., increased heart rate, increased breathing, increased perspiration) and/or a subjective measure of stress provided by the target individual. The intensity of stress, the length of time the stressful event lasts, and/or time of day of the stressful event.
Result of reaction-time game. The reaction-time game may be presented within the GUI, for example, at defined time intervals, at predefined events, and/or triggered by events, for example, after multiple activities have occurred. The reaction-time game measures a reaction time of the target individual in response to visual and/or auditory stimulus. For example, the user may be asked to press a button in the screen as fast as possible after hearing a certain sound, and/or after seeing a certain displayed image.
Energy state for the target individual, which may be predicted, calculated, and/or provided by the target individual as an answer to a question. Energy state may be obtained, for example, at wake-up, and/or at one or more times throughout the day.
Motivation. Optionally, a perception of motivation, which may be manually entered by the user via the user interface. The amount of motivation may be entered at defined time intervals and/or at trigger events.
Mood. Optionally, a perception of mood, which may be manually entered by the user via the user interface. The type of mood may be entered at defined time intervals and/or at trigger events.

One or more sleep parameters may be automatically computed according to data of one or more sensors. Exemplary sensors include physiological sensors that measure one or physiological parameters of the target individual, and/or activity sensors that measure one or more activities of the target individual.

Exemplary activity sensors include: a microphone of the client terminal (e.g. smartphone) that senses noise such as snoring or lack or noise indicating sleep or senses the voice of the target individual indicating lack of sleep, a camera of the client terminal (e.g., smartphone) that capture images (e.g., video) of the target individual sleep and code that analyzes the images to compute the sleep-parameters, a location-based sensor (e.g., GPS) that senses the geographic location of the target individual, a mobility sensor that determines whether the target user is still (i.e., in bed sleeping or trying to sleep) or walking around, a bed-movement sensor that determined whether the target individual is lying still in bed or is moving around in bed (e.g., restless sleep, tossing and turning) such as an accelerometer.

Exemplary physiological sensors include: a heart rate sensor that measures heart rate, an eye state sensor that measures whether the eye is open or closed and/or eye movements, a breathing sensor that measures breathing rates, and a brain signal sensor that measures brain signals (e.g., EEG).

The output of the activity and/or physiological sensor(s) may be analyzed to identify when the target individual is sleeping, the state of sleep (e.g., REM, deep sleep, light sleep), when the target individual is awake, whether the target individual is in bed trying to sleep, and/or whether the target individual is experiencing high levels of stress. For example, sleep or awake states may be estimated according to heart rate, breathing rate, and brain signals. Total time trying to sleep may be estimated from output of the mobility sensor.

Sensors may be wearable, incorporated into an object worn by the target individual, for example, a watch, a necklace, a chest belt, a ring, socks, pants, shoes, undergarments, a wrist band, a head band, a smart shirt, a wall mounted sensor, and a hat. Exemplary wearable sensors include: a heart rate sensor that senses heart rate, a movement sensor that tracks steps (i.e., walking, running), an activity sensor that senses fitness activities, a calorie sensor that estimates calories being burnt, a temperature sensor that senses body temperature, a perspiration sensor that senses perspiration, a pulse oximeter that senses hemoglobin oxygenation levels, a breathing sensor that senses respiratory rate, and an electrogram sensor that measures electrical activity of tissue (e.g., heart, brain, and muscle).

Sensors may be contactless sensors that do not directly contact the target individual, for example, indirectly contacting the target individual, for example, located under the mattress, located on the surface of the mattress, and located within a bag and/or purse being carried by the target individual. Such sensors may be implemented, for example, as accelerometers, location based sensor, microphone, and/or camera of a Smartphone.

Sensors may be internet of things (IoT) enabled, which may be implemented within household items, for example, within a coffee machine to transmit indications of when the target individual user is drinking coffee, code that analyzes security surveillance videos to determine the location and/or activity of the target individual within the home, a smart-TV that transmits indications of when the target individual is watching television, and a IoT enabled treadmill that transmits indications of when the target individual is exercising.

One or more sleep parameters may be computed according to perceived data entered by the user via the GUI. For example, the target user marks on a scale (e.g., from 1 to 10) an indication of sleep satisfaction, day time sleepiness, and/or daytime stress.

As used herein, the general assumption is that people sleep at night and are awake during the day, however, the systems, methods, apparatus, and/or code instructions described herein are not necessarily limited to day and night, and are applicable to other scenarios where people are awake at night and sleep during the day (e.g., shift workers), are awake for extended periods of time that include night and day (e.g., on call physicians, soldiers, researchers in the arctic and/or Antarctica), and/or experience abnormal lengths of night and/or day (e.g., plane travelers).

At 208, additional data of the user may be accessed. The additional data may be for the recent sleep session, such as a sleep state of the user. The additional data may be for multiple previous sleep sessions, such as user data that remains constant and/or that slowly changes, such as characteristics of the user.

The sleep-state may indicate a state of the sleep condition of the target individual. The sleep-state may be obtained as an outcome of a sleep application. The sleep-application may be selected from multiple sleep applications. Examples of sleep applications include: a sleep evaluation application, a sleep improvement application, a sleep monitoring application, and a sleep maintenance application. The sleep application may be selected, for example, as a default, by the user, by a sleep expert, and/or automatically (e.g., as an outcome of a sleep application selection machine learning model in response to an input of sleep-parameters and/or other data of historical sleep sessions preceding the recent sleep session, where the sleep application selection machine learning model may be trained on a training dataset of sample sleep-parameters obtained for a plurality of sample individuals each labelled with a ground truth indication of a certain sleep application selected from the plurality of sleep applications).

The sleep-state may be an indication of the sleep condition.

The set of characteristics of the target user may denote the target user's daytime behavior and/or demographic parameters.

Exemplary characteristics may indicate the target user's sleep behavior and/or history and/or demographic parameters of the target user. Examples of the characteristics of the user include attributions, descriptors, labels, symptoms, tendencies, or other values which define the user's sleep behavior and history, history of sleep issues and/or disturbances and/or symptoms of such issues, demographic information such as age, BMI, medical conditions (other than sleep disorders, such as heart disease, diabetes), occupation, geographical location, gender and more, class or label obtained from a different classification process, etc.

The set of characteristics and/or the sleep-parameters and/or other data may be provided as input to the sleep application for generating the sleep-state.

The additional data may include other data previously obtained as outcomes of the machine learning model in previous iterations, for example, presentation format(s) for presentation of the outcome (e.g., instructions).

The additional data may include monitored interaction of the target user with the previously provided outcome (e.g., instructions) of the previous iteration (e.g., as described with reference to 218).

At 210, features may be extracted from the data.

Features may be extracted by applying various functions, which may be, for example, hand crafted features, and/or features automatically learned by other trained machine learning models, such as encodings from trained neural networks. Features may be identified and extracted using automated feature identification and/or extraction approaches, such as brute force and/or heuristic approaches that test different combinations of features and select the most relevant ones.

The features may be customized by being selected according to the set of characteristics of the target user. Features may be selected, for example, by a mapping dataset, set of rules, and/or other approaches (e.g., trained models) that maps characteristics to features. For example, features relevant to certain characteristics are selected, while other features not relevant to certain characteristics are not selected. For example, people living in cold climates may require body temperature as a feature (e.g., being cold at night may impact sleep), while people living in moderate climates may not require body temperature as a feature since the body temperature is assumed to be normal and therefore not impacting on sleep.

Weights may be computed for respective sleep-parameters and/or respective features, for example, according to the characteristics of the user. Weights may be set, for example, by a mapping dataset, set of rules, and/or other approaches (e.g., trained models) that compute weights based on features. For example, people that are obese may be at increased risk of sleep apnea, and features related to sleep apnea may be assigned higher weights. Older males may be at increased risk for enlarged prostate, and feature related to urinary frequency may be assigned higher weight. For people working stressful jobs, feature related to stress and/or coffee consumption may be assigned higher weights.

Features may be extracted from the sleep-parameters and/or from other data.

Exemplary features include:
Total nights tracked by the target individual in a period of time (e.g., previous week), with a sleep application.
Longest streak of consecutive nights tracked by the target individual with a sleep application.
Number of days passed since the target individual last tracked sleep with a sleep application.
A list of in-app tools and capabilities available for target individual in the sleep application, at a given time.
A moving trend of interactions of the target individual with the sleep application.
Time that the target individual spent reading sleep-related articles, viewing sleep-related educational videos, listening to sleep-related information audio, etc., as instructed by a sleep application, in a period of time (e.g., in the past week).
Indications for associations found between sleep-related habits (e.g., caffeine consumption prior to bedtime) and poor sleep quality and/or duration.
Indications for periodicity of sleep parameters, e.g., target individual goes to sleep later on a specific day of the week.
An indication on whether at least one of several sleep parameters were lower or higher than a set of thresholds, e.g., sleep latency was longer than 30 minutes or sleep efficiency was lower than 85%.
Average weekly bedtime.
Discrepancies between total time spent in bed and required time in bed for user.
A sleep-parameter (e.g., raw, without further processing) may be used as a feature.

At 212, data obtained from one or more of 204-210 is fed into the machine learning model. Data fed into the model may be from a single previous sleep session, and/or from multiple previous sleep sessions. Data from multiple previous sleep sessions may be arranged in a sequence and/or labelled with a date, indicating relative arrangement of the data according to previous nights. Data of more recent nights may be assigned higher weights relative to data from earlier nights, for example, indicating that the data of more recent nights is more relevant. For example, recent habits (e.g., recent coffee consumption, sleep schedule) reflect the current reality more than older habits which may not be relevant.

The sleep-parameters (accessed in 206) and/or the previously generated outcomes, optionally instructions (accessed in 204) are fed into the machine learning model.

Optionally, the additional data is fed into the machine leaning model, optionally in combination with the sleep-parameters and/or the previously generated instructions, such as the sleep-state and/or the set of characteristics of the user (accessed in 208).

Alternatively or additionally, the extracted features (obtained in 210) are fed into the machine learning model.

Exemplary architectures of the machine learning model include, for example, statistical classifiers and/or other statistical models, neural networks of various architectures (e.g., convolutional, fully connected, deep, encoder-decoder, recurrent, graph), support vector machines (SVM), logistic regression, k-nearest neighbor, decision trees, boosting, random forest, a regressor, and/or any other commercial or open source package allowing regression, classification, dimensional reduction, supervised, unsupervised, semi-supervised or reinforcement learning. Machine learning models may be trained using supervised approaches and/or unsupervised approaches.

Alternatively or additionally, the machine learning model is implemented using one or more features described with reference to FIG. 4, for which a summary is provided as follows:

The data being fed into the machine learning model may be run through one or more decision trees. Each decision tree includes a set of predefined rules and/or a sub-processing code. Each decision tree terminates in a respective neuron that outputs an active category of a binary indication when conditions in the respective decision tree are satisfied. The respective neuron may be mapped to output(s) that generate(s) the outcome (e.g., the instructions) for improving the sleep condition.

A neuron may be implemented as a Boolean parameter (having a binary value, such as 0/1, or TRUE/FALSE). Each neuron may be located at the end node of a decision tree, i.e., its value is changed from 0 to 1 (the neuron is “lit”) if, and only if, all conditions in the decision tree's branches were satisfied along the way to this end node. Each neuron may be mapped to a set of outputs (OP). A neuron may be mapped, e.g., to a set of OPs which address associations found between user's sleep quality and user's daily amount of consumed caffeine.

For example, consider a decision tree for a sleep improvement program. The first condition in such a tree is whether the target individual is indeed taking part in such a program. From there, different branches may be used depending on the specific sleep difficulties target individual experienced, as evaluated by a sleep evaluation program, e.g., tendencies of insomnia accompanied by bad sleep habits. Additional branches may then be determined based on the specific type of instructions, or “challenge”, individual target received as part of the sleep improvement program, e.g., an instruction to stay in bed for no more than 7 hours per night and to avoid drinking coffee in the evening. Additional branches of this decision tree may be chosen depending on target individual's performances and ability to comply with said instructions, e.g., target individual was able to restrict bedtime to 7 hours according to instructions but failed to abstain from evening coffee. The end node of such a branch may result in lighting a neuron specific for this situation:

Target individual is in a sleep improvement program, AND

Target individual was evaluated as experiencing symptoms of insomnia and bad sleep habits, AND

Target individual was instructed to restrict bedtime and to avoid evening coffee, AND

Target individual successfully restricted bedtime, AND

Target individual was not successful in avoiding evening coffee.

A neuronal group (NG) may be implemented as a set of neurons congregated based on a characteristic and/or a set of characteristics which define the group, e.g., a group of all neurons mapped to OPs which address associations found between user's sleep quality and all user's daily food- and drink-related habits.

A basket may be implemented as a set of NGs congregated based on a characteristic and/or a set of characteristics which can define the basket, e.g., a group of all NGs containing neurons mapped to OP which relate to user's sleep improvement program.

Neurons that output the active category are multiplied by a set of weights to obtain a weighted set of active neurons. The set of weights may be, for example, prefixed, determined by a weighting function that receives data fed into the machine learning model as input, and/or previously learned. The set of weights may be defined per neuron, defined per neuronal group (i.e., a set of neurons congregated based on a certain characteristic and/or set of characteristics), per basket (i.e., a set of neuronal groups congregated based on a certain characteristic and/or set of characteristics), and combinations of the aforementioned.

An order of the weighted neurons in the weighted set may be randomized.

Each respective weighted neuron may be penalized based on neurons that were activated during a previous feeding iteration of the machine learning model (i.e., that generated the outcome of the previously generated instructions). The respective neuron may be penalized by computing a penalty function, which may be a function of time (and/or iterations) passed since the activation of the respective neuron, and not necessarily whether activation occurred in the previous feeding iteration. For example, when a respective individual weighted neuron was activated 6 days ago, the neuron may be penalized, despite not being activated in the previous feeding iteration.

Penalties computed for each respective weighted neuron may be aggregated to obtain a respective total neuron-penalty. A transfer function may be applied on each respective total neuron-penalty.

The penalizing may be based on one or more of:

(i) whether a respective individual weighted neuron was previously activated, for example, during the (one) previous iteration (i.e., most recent), during one or more earlier iterations prior to the most recent and not during the most recent, as a function of time passed since activation, and/or a number of iterations since the activation,

(ii) whether a neuronal group of which the respective individual weighted neuron is a member, includes neurons that were previously activated, for example, during the (one) previous iteration (i.e., most recent), during one or more earlier iterations prior to the most recent and not during the most recent, as a function of time passed since activation, and/or a number of iterations since the activation,

(iii) whether the respective individual weighted neuron that was previously activated is of a same output format as the instructions (e.g., push notification, audio file, video file, image, etc.), for example, during the (one) previous iteration (i.e., most recent), during one or more earlier iterations prior to the most recent and not during the most recent, as a function of time passed since activation, and/or a number of iterations since the activation generated a same output format of the instructions, and

(iv) applying one or more additional penalties defining the outcome (e.g., of the instructions).

For each basket of a set of neuron groups each of a set of neurons, a first top set of neurons within lowest penalty values may be selected according to a first requirement. From the selected first top set of neurons, for each neuron group, a second top set of neurons within lowest penalty values may be selected according to a second requirement. From the selected second top set of neurons, a third top set of neurons within lowest penalty values may be selected according to a third requirement.

The outcome (e.g., of the instructions) may be generated according to the third top set of neurons.

At 214, outcome of the machine learning model may be obtained. The outcome may be a direct output of the machine learning model, for example, a last layer of a neural network.

The outcome may be generated by an aggregation of weights of internal settings of the machine learning model set according to the feeding, and penalty functions applied to the weights. Alternatively or additionally, the outcome (e.g., of the instructions) may be generated according to the third top set of neurons.

The outcome may be instructions for presentation on a user interface (e.g., text, audio, video, and the like) indicating treatment for improvement of the sleep condition of the target individual for a new sleep session following the recent sleep session, for example, for an upcoming night following a previous night. Outcomes may be categorized based on output type, e.g., push notifications, emails, voice messages.

Optionally, a presentation format is obtained as an outcome of the machine learning model. Exemplary candidate presentation formations for presenting the instructions for improving the sleep condition include: push notification, email, voice message, video message, animation, presentation, and text message. During a next iteration, the presentation format outcome may be fed into the machine learning model.

The machine learning model may learn which presentation format is most impactful on the user, such as which presentation format results in the highest improvement in the sleep-state of the user. For example, some users may respond better to audio, while other users may respond better to video.

Examples of instructions to treat the sleep condition include:
Get more hours of sleep.
Reduce caffeine intake throughout the day.
Reduce or stop fluid intake 3 hours before going to bed.
Change sleep schedule, for example, go to sleep at 10 PM rather than midnight, and wake up at 6 AM.
Exercise during the day, but not during the 3 hours before going to bed.
Change pillow and/or mattress to increase comfort.
Use earplugs to reduce noise.

The instructions may be for other cases (i.e., not necessarily for a sleep disorder that is to be improved), for example:
For a target individual who is a healthy sleeper and wish to gain additional insights into her sleep.
For a target individual who is not suffering from a sleep disorder but does experience some sleep difficulties.
For a target individual who is suffering from a sleep disorder.
For a target individual who suffered from a sleep disorder or did not suffer from a sleep disorder but did experience some sleep difficulties in the past, and is now maintaining healthy sleep, as part of a relapse prevention program.
As part of a sleep evaluation program.
As part of a sleep improvement program, for a target individual who suffers from sleep disorders.
As part of a sleep improvement program, for a target individual who does not suffer from sleep disorders but does experience some sleep difficulties.
As part of a sleep maintenance and/or relapse prevention program.
As a tool for target individuals who are healthy sleepers and wish to gain insights into their sleep.

In an example, a user is assessed by a sleep assessment algorithm as suffering from symptoms of insomnia. The user is then directed to a sleep improvement program, which is structured as short- and long-term tasks given to the user in order to alleviate symptoms of insomnia and improve sleep quality. The sleep improvement program may access the outcomes of the machine learning model described herein to select messages which will be sent to the user to encourage the user to comply with certain tasks, celebrate the user's achievements, emphasize the importance and meaning of the certain tasks, deliver personalized tasks, and more.

At 216, the outcome, optionally instructions, for improving the sleep-state may be presented to the user, optionally using the presentation format outputted by the machine learning model. The outcomes may be communicated to the user, for example, using one or more of: push notifications, emails, voice messages, video messages, animations, presentations, textual messages.

Alternatively or additionally, the outcome may be, for example, stored on a local storage device, forwarded to a remote device (e.g., server used by a sleep evaluation expert), and/or provided to another process as input (e.g., fed into a sleep application).

At 218, interaction of the target user may be monitored. The interaction may be monitored after the instructions and/or other outcome of the machine learning model is presented to the user. For example, interaction of the user with the sleep application and/or with a user interface (e.g., graphical user interface (GUI), video, screen, mobile device, microphone, and the like) presenting the instructions. For example, whether the user replays the instructions, whether the user clicks on icons and/or links, and/or whether the user searches and/or accesses additional information. The monitoring may be of the same display, such as a display of a smartphone, when the same display is used to present the instructions and to access a sleep application. The monitoring may be of different displays, such as when a display of a smartphone is used to present the instructions, and a display of a tablet is used to access the sleep application.

The monitored interaction of the target user may be fed into the machine learning model during the next iteration.

At 220, one or more features described with reference to 202-218 may be iterated, for example, per sleep session, or per multiple sleep session, such as per night, or every 3 nights, or every week, or other interval.

During iterations, the machine learning model may be dynamically updated in 202. A new set of sleep-parameters may be accessed for the new sleep session, and/or other new data may be obtained, for feeding into the machine learning model in combination with the instructions for the new sleep session. Another set of instructions for another sleep session following the new sleep session is obtained. A new sleep-state is accessed for the new sleep session from a new selection of the sleep application, for feeding into the machine learning model.

Referring now back to FIG. 3, at 302, sample sleep-parameters are obtained for a sample individual. The sample sleep-parameters is obtained at least for a recent sleep session. Sleep-parameters may be obtained for one or more historical sleep sessions that are earlier than the recent sleep session.

At 304, previously generated sample outcomes of the machine learning model, such as instructions for improving sleep for the recent sleep session, as obtained. The outcomes may be obtained, for example, as described with reference to FIG. 2. It is noted that initially, there may not be any outcomes of the machine learning model, in which case the instructions may be manually provided (e.g., by a sleep expert), and/or automatically generated by a sleep application (which may not necessarily be a machine learning process).

At 306, other sample data may be obtained, for example, one or more of:
A sleep-state indicating a state of a respective sleep condition of the respective sample individual, for example, as described herein.
The monitored interaction of the target user, for example, as described herein, for example, as described herein.
The presentation format outcome, for example, as described herein.
Extracted features, for example, as described herein.
Set of characteristics of the target user, for example, as described herein.

At 308, records are obtained for multiple sample individuals, by iterating 302-306 for each sample individual.

At 310, each record is labelled with a ground truth indication of instructions for improving the respective sleep condition of the respective sample individual during a subsequent sleep session. The instructions may be obtained, for example, manually provided (e.g., by a sleep expert), and/or automatically generated by a sleep application.

At 312, a training dataset that includes the labelled records is generated.

At 314, the machine learning model is trained on the training dataset.

The machine learning model is trained for generating an outcome, such as instructions for presentation on a user interface indicating treatment for improvement of the target sleep condition of the target individual for a new sleep session following the recent sleep session, in response to an input of sleep-parameters and previously generated instructions, and/or other data described herein.

At 316, one or more of 302-314 may be iterated.

The machine learning model may be iteratively trained and/or updated by updating the records using a new set of sleep-parameters accessed for the new sleep session, using the instructions for the new sleep session obtained as outcomes of the machine learning model for the recent sleep session, and/or using other newly obtained data.

Referring now back to FIG. 4, at 402, the process starts.

At 404, input data is accessed and/or processed. For example, one or more of:
Previously generated sample outcomes of the machine learning model, for example, as described herein.
Sleep-parameters of the target individual obtained for a previous sleep session, optionally for multiple historical sleep sessions, for example, as described herein.
The monitored interaction of the target user, for example, as described herein.
The presentation format outcome, for example, as described herein.
Extracted features, for example, as described herein.
Set of characteristics of the target user, for example, as described herein.

Features may be extracted from the input data, optionally from the sleep-parameters, for example, as described herein.

At 406, external services and/or processes may be employed to extract information from data, optionally the input data, for example, a sleep-state indicating a state of a respective sleep condition of the target individual may be obtained from an external sleep application, as described herein.

At 408, the processed data may be run through a series of decision trees (DT). The DTs may be predefined as a list of rules and/or generated by an external process. Each end node in DT controls a neuron, which is lit when (e.g., only if) the end node's condition is met. Each neuron is mapped to a set of outputs (OP).

At 410, a set of weights (W) may be predefined and/or determined by a weighting function (ƒ_{w}). The set of weights W and/or the weighting function ƒ may be defined per neuron, and/or per neuronal group (NG), and/or per basket, and/or as a combination thereof. The weighting function ƒ_{w} may consider all input data described herein, e.g., sleep data, user characteristics, user's system states data, usage and engagement data, previous outcomes during a previous iteration (PO), and more.

At 412, the lit neurons (N), optionally all lit neurons are gathered (e.g., aggregated) and multiplied by the set of weights (W), to obtain a weighted array of lit neurons (WN).

At 414, the order of the weighted neurons in WN is randomized.

At 416, a penalty array (NPA), which holds a penalty value for each of the weighted neurons in WN, may be initialized.

At 418A-D, each weighted neuron (wn) in WN may be penalized, based on previously selected neurons in time (τ), as given in PO. The penalty may be positive and/or negative. Penalties may be based on one or more of:

At 418A, based on a neuron, i.e., if the same neuron wn was previously selected as an output, by applying a penalty function denoted ƒₙₚ(wn,τ).

Alternatively or additionally, at 418B, based on neuronal groups (NG), i.e., if previously selected neurons were associated with the same NG to which the neuron wn is associated to, by applying a penalty function denoted ƒ_{ngp}(wn,τ)

Alternatively or additionally, at 418C, based on output type, i.e., if previously selected neurons were the same type as the neuron wn, by applying a penalty function denoted ƒₒₜ(wn,τ).

Alternatively or additionally, at 418D, based on additional characteristics, and/or features, of specific outputs in OP, when required, by applying additional penalty functions. For example, an output which should only be selected a predefined number of times, an output relevant only for a specific cohort of users, etc.

At 420, penalties (e.g., all penalties) for each neuron wn=WN(i) are added to get the corresponding total penalty value in NPA(i).

At 422, a transfer function denoted ƒ_{tw}(npa) is applied on each penalty value (npa) in NPA to utilize neuron weights in WN.

At 424, for each basket, the top K_{basket} neurons with the lowest penalty values are selected.

At 426, from the resulting set of neurons, for each NG, the top K_{NG} neurons with the lowest penalty values are selected.

At 428, from the resulting set of neurons, the top K_{N} neurons with the lowest penalty values are selected. The selected neurons are the outcome of the machine learning model described herein.

At 430, the process ends when the output, i.e., the outcome is provided.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant machine learning models will be developed and the scope of the term machine learning model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A computer implemented method of generating outcomes for treatment for improving a sleep condition in a target individual, comprising:
iterating over a plurality of sleep sessions:
accessing a plurality of previously generated instructions for treatment for improvement of the sleep condition for a recent sleep session obtained as a previous outcome of a machine learning model;
accessing a plurality of sleep-parameters computed for the recent sleep session;
feeding the plurality of sleep-parameters and the plurality of previously generated instructions into the machine learning model; and
obtaining as an outcome of the machine learning model, instructions for presentation on a user interface indicating treatment for improvement of the sleep condition of the target individual for a new sleep session following the recent sleep session;
wherein a new set of plurality of sleep-parameters are accessed for the new sleep session, for feeding into the machine learning model in combination with the instructions for the new sleep session, for obtaining another set of instructions for another sleep session following the new sleep session.

2. The computer implemented method of claim 1, wherein the outcome is generated by an aggregation of weights of internal settings of the machine learning model set according to the feeding, and penalty functions applied to the weights.

3. The computer implemented method of claim 1, further comprising:
accessing a sleep-state for the recent sleep session, the sleep-state indicating a state of the sleep condition of the target individual obtained as an outcome of a sleep application selected from a plurality of sleep applications; and
wherein feeding further comprises feeding the sleep-state into the machine learning model,
wherein a new sleep-state is accessed for the new sleep session from a new selection of the sleep application, for feeding into the machine learning model.

4. The computer implemented method of claim 3, wherein the sleep application is selected from a group consisting of: a sleep evaluation application, a sleep improvement application, a sleep monitoring application, and a sleep maintenance application.

5. The computer implemented method of claim 1, further comprising monitoring interaction of the target user with the sleep application and/or with a graphical user interface (GUI) presenting the instructions, and wherein feeding further comprises feeding into the machine learning model, the previously monitored interaction of the target user.

6. The computer implemented method of claim 1, wherein obtaining comprises obtaining as the outcome of the machine learning model, a presentation format selected from a plurality of candidate presentation formats, for presenting the instructions for improving the sleep condition, selected from a group consisting of: push notification, email, voice message, video message, animation, presentation, and text message, wherein feeding further comprises feeding the previously obtained presentation format into the machine learning model.

7. The computer implemented method of claim 1, wherein the machine learning model is trained on a training dataset that includes records for a plurality of sample individual, each record for each respective sample individual including sample sleep-parameters and previously generated instructions for improving sleep for a recent sleep session labelled with a ground truth indication of instructions for generating outcomes for improving a respective sleep condition of the respective sample individual during a new sleep session.

8. The computer implemented method of claim 1, further comprising extracting a plurality of features from the plurality of sleep-parameters, wherein the plurality of features are fed into the customized machine learning model and/or used by the sleep application to generate the sleep-state.

9. The computer implemented method of claim 1, further comprising accessing a set of characteristics of the target user denoting the target user's daytime behavior and/or demographic parameters, and wherein feeding further comprises feeding into the machine learning model, the set of characteristics.

10. The computer implemented method of claim 1, wherein feeding into the machine learning model comprises running data fed into the machine learning model through a plurality of decision trees each comprising a set of predefined rules and/or a sub-processing code, wherein each decision tree terminates in a respective neuron that outputs an active category of a binary indication when conditions in the respective decision tree are satisfied, the respective neuron is mapped to at least one output that generates the outcome of the instructions for improving the sleep condition.

11. The computer implemented method of claim 10, further comprising:
multiplying neurons that output the active category by a set of weights to obtain a weighted set of active neurons,
wherein the set of weights are at least one of: prefixed, determined by a weighting function that receives data fed into the machine learning model as input, and previously learned;
wherein the set of weights are at least one of: defined per neuron, defined per neuronal group comprising a set of neurons congregated based on a certain characteristic and/or set of characteristics, per basket of a set of neuronal groups congregated based on a certain characteristic and/or set of characteristics, and combinations of the aforementioned.

12. The computer implemented method of claim 11, further comprising randomizing an order of the weighted neurons in the weighted set.

13. The computer implemented method of claim 11, further comprising:
penalizing each respective weighted neuron based on neurons that were activated during a previous feeding iteration of the machine learning model that generated the outcome of the previously generated instructions;
aggregating penalties computed for each respective weighted neuron to obtain a respective total neuron-penalty; and
applying a transfer function on each respective total neuron-penalty.

14. The computer implemented method of claim 13, wherein the penalizing is based on a member selected from a group consisting of:
(i) whether a respective individual weighted neuron was previously activated,
(ii) whether a neuronal group of which the respective individual weighted neuron is a member, includes neurons that were previously activated,
(iii) whether the respective individual weighted neuron that previously activated generated a same output format of the instructions, and
(iv) applying at least one additional penalty defining the outcome of the instructions.

15. The computer implemented method of claim 13, further comprising:
for each basket of a set of neuron groups each of a set of neurons, selecting a first top set of neurons within lowest penalty values according to a first requirement;
from the selected first top set of neurons, for each neuron group, selecting a second top set of neurons within lowest penalty values according to a second requirement;
from the selected second top set of neurons, selecting a third top set of neurons within lowest penalty values according to a third requirement,
wherein the outcome of the instructions is generated according to the third top set of neurons.

16. A computer implemented method of training a machine learning model for generating outcomes for treatment for improvement of a sleep condition in a target individual, comprising:
obtaining records for each of a plurality of sample individuals, each record including sample sleep-parameters, previously generated instructions for treatment for improvement of a respective sleep condition for a recent sleep session, and a sleep-state indicating a state of a respective sleep condition of the respective sample individual;
labelling each record with a ground truth indication of instructions for improving the respective sleep condition of the respective sample individual during a subsequent sleep session;
generating a training dataset that includes the labelled records; and
training the machine learning model on the training dataset for generating instructions for presentation on a user interface indicating treatment for improvement of a target sleep condition of a target individual for a new sleep session following a recent sleep session in response to an input of sleep-parameters and previously generated instructions,
wherein the machine learning model is iteratively trained by updating the records using a new set of sleep-parameters accessed for the new sleep session, and using the instructions for the new sleep session obtained as outcomes of the machine learning model for the recent sleep session.

17. A system for generating outcomes for treatment for improvement of a sleep condition in a target individual, comprising:
at least one hardware processor executing a code for:
iterating over a plurality of sleep sessions:
accessing a plurality of previously generated instructions for treatment for improvement of the sleep condition for a recent sleep session obtained as a previous outcome of a machine learning model;
accessing a plurality of sleep-parameters computed for the recent sleep session;
feeding the plurality of sleep-parameters and the plurality of previously generated instructions into the machine learning model; and
obtaining as an outcome of the machine learning model, instructions for presentation on a user interface indicating treatment for improvement the sleep condition of the target individual for a new sleep session following the recent sleep session;
wherein a new set of plurality of sleep-parameters are accessed for the new sleep session, for feeding into the machine learning model in combination with the instructions for the new sleep session, for obtaining another set of instructions for another sleep session following the new sleep session.
